(19) United States
(12) Patent Application Publication
(10) Pub. No.: US 2022/0071563 A1
(43) Pub. Date: Mar. 10, 2022

(54) Wearable health monitoring system

(71) Applicant: LEDO Network, Inc., Cupertino, CA (US)

(72) Inventors: Bryan He Huang, Cupertino, CA (US); Shiyou Ai, Cupertino, CA (US)

(21) Appl. No.: 17/014,657

(22) Filed: Sep. 8, 2020

(51) Int. Cl. A61B 5/00; A61B 5/0205

(52) U.S. Cl. CPC A61B 5/6808; A61B 5/02438; A61B 5/4812; A61B 5/746; A61B 5/7282; A61B 5/02055; A61B 5/0022; A61B 5/7285; A61B 5/4818; A61B 2562/0219; A61B 2560/0252; A61B 2562/029; A61B 2503/04; A61B 2562/0223; A61B 5/4809

(57) ABSTRACT

A method for monitoring health of a person by a health monitoring system includes determining if a person wearing a wearable health monitoring device is sleeping or not based on movement data, measuring sleep movements of the person to determine whether the person is in a deep sleep mode when the person is determined to be sleeping, measuring bio-vital signals of the person and at least one of a posture of the person or environmental parameters when the person is determined to be in the deep sleep mode, and automatically producing an alarm signal if a predetermined criterion is met based on the bio-vital signals and at least one of a posture of the person or the environmental parameters.

### BACKGROUND OF THE INVENTION

The present invention relates generally to the field of healthcare technologies, and in particular, to a wearable device and a related system for monitoring health of a person such as a baby.

Every year tens of thousands of babies die from Sudden Infant Death Syndrome (“SIDS”) around the world. While specific causes of SIDS might be difficult to determine, the major causes can be categorized as following: 1) inappropriate sleeping positions, such as stomach sleeping causing cessation of breathing; 2) inappropriate layers of clothing or environmental temperature causing baby overheating or getting too cold; and 3) other unknown reasons that may cause cessation of breathing. Nowadays, many parents are still using a video camera or an audio monitor to watch or listen to their babies, for motion or audio detection. These systems, however, may not provide a parent with enough information to intervene before a serious health issue happens to the baby.

There is therefore a need for an improved health monitoring system to address the above described problems.

### SUMMARY OF THE INVENTION

In one general aspect, the present invention relates to a method for monitoring health of a person by a health monitoring system, comprising: determining, based on movement data, if a person wearing a wearable health monitoring device is sleeping or not, wherein the movement data is produced by one or more movement sensors in the wearable health monitoring device; when the person is determined to be sleeping, measuring sleep movements of the person to determine whether the person is in a deep sleep mode; measuring one or more bio-vital signals of the person and at least one of a posture of the person or one or more environmental parameters when the person is determined to be in the deep sleep mode, wherein the one or more bio-vital signals are produced by one or more bio-vital signal detectors in the wearable health monitoring device; and automatically producing an alarm signal if a predetermined criterion is met based on the one or more bio-vital signals and at least one of a posture of the person or the one or more environmental parameters.

Implementations of the system may include one or more of the following. The one or more bio-vital signals can include a respiration rate. The method can further include: automatically determining by a health monitoring system whether the person has a slow respiration or a fast respiration by comparing the respiration rate to a predetermined respiration threshold, wherein the health monitoring system includes the wearable health monitoring device. The method of claim can further include when the person is determined to have a slow respiration, automatically producing the alarm signal if the respiration rate is below a critical respiration threshold. The method can further include: when the person is determined to have a slow respiration, automatically determining a posture of the person based on measurement by an accelerometer sensor in the wearable health monitoring device, wherein the alarm signal is produced if the person is determined to be sleeping on stomach based on the posture of the person. The method can further include: when the person is determined to have a fast respiration, automatically determining whether respiration behaviors are within a safe zone based on an absolute value of the respiration rate and a period of time within which the respiration rate is above the predetermined respiration threshold; and automatically producing an alarm signal if the respiration behaviors are outside of the safe zone. The respiration behaviors are outside of the safe zone when the respiration behaviors are above a safe respiration threshold, or the person is determined to have a fast respiration for an extended period long than a threshold period, or a combination thereof. The method can further include: when the person is determined to have a fast respiration, measuring one or more environmental parameters; automatically determining by a health monitoring system whether the one or more environmental parameters are within respective desirable ranges, wherein the health monitoring system includes the wearable health monitoring device, wherein the alarm signal is produced if the one or more environmental parameters are determined to be outside of respective desirable ranges. The one or more environmental parameters can include ambient temperature or humidity. The person can be an infant, baby, or toddler. The wearable health monitoring device can be attached to or removably disposed in a wearable article worn by and in contact with the baby's abdomen. The one or more movement sensors in the wearable health monitoring device can include one or more of an accelerator, a magnetic detector, a digital compass, a gyroscope, a pressure sensor, an inertia module, or a piezoelectric sensor. The one or more bio-vital signal detectors in the wearable health monitoring device can include one or more of a body temperature sensor, a respiratory sensor, a blood pulse sensor, a blood oxygen sensor, or one or more electric signal sensors.

In another general aspect, the present invention relates to a health monitoring system that includes a wearable health monitoring device comprising one or more movement sensors that can produce movement data of a person that wears the wearable health monitoring device, wherein the wearable health monitoring device includes one or more bio-vital signal detectors that can produce one or more bio-vital signals; and one or more computer processor that can determine, if the person is sleeping or not based on movement data, wherein the one or more movement sensors can measure sleep movements of the person when the person is determined to be sleeping, wherein the one or more computer processors can determine whether the person is in a deep sleep mode, wherein when the person is determined to be in the deep sleep mode, the one or more computer processors can produce an alarm signal if a predetermined criterion is met based on the one or more bio-vital signals and at least one of a posture of the person.

Implementations of the system may include one or more of the following. The one or more bio-vital signals include a respiration rate, wherein the one or more computer processors can automatically determine whether the person has a slow respiration or a fast respiration by comparing the respiration rate to a predetermined respiration threshold. When the person is determined to have a slow respiration, the one or more computer processors can produce the alarm signal if the respiration rate is below a critical respiration threshold. The wearable health monitoring device includes an accelerometer, wherein when the person is determined to have a slow respiration, the one or more computer processors can automatically determine a posture of the person based on measurement by the accelerometer, wherein the alarm signal is produced if the person is determined to be sleeping on stomach based on the posture of the person. When the person is determined to have a fast respiration, the one or more computer processors can automatically determine whether respiration behaviors are within a safe zone based on an absolute value of the respiration rate and a period of time within which the respiration rate is above the predetermined respiration threshold, wherein the one or more computer processors can automatically produce an alarm signal if the respiration behaviors are outside of the safe zone. The respiration behaviors are outside of the safe zone when the respiration behaviors are above a safe respiration threshold, or the person is determined to have a fast respiration for an extended period long than a threshold period, or a combination thereof. The health monitoring system can further include one or more environmental sensors configured to produce one or more environmental parameters, when the person is determined to be in the deep sleep mode, the one or more computer processor can further produce an alarm signal if a predetermined criterion is met based on the one or more bio-vital signals and at least one of a posture of the person or one or more environmental parameters. When the person is determined to have a fast respiration, one or more environmental sensors can measure one or more environmental parameters, wherein the one or more computer processors can automatically determine whether the one or more environmental parameters are within respective desirable ranges, wherein the one or more computer processors can automatically produce the alarm signal if the one or more environmental parameters are determined to be outside of respective desirable ranges, wherein the one or more environmental parameters include ambient temperature or humidity. The person is an infant, baby, or toddler, wherein the wearable health monitoring device is attached to or removably disposed in a wearable article worn by and in contact with the baby's abdomen. The one or more movement sensors include one or more of an accelerator, a magnetic detector, a digital compass, a gyroscope, a pressure sensor, an inertia module, or a piezoelectric sensor. The one or more bio-vital signal detectors include one or more of a body temperature sensor, a respiratory sensor, a blood pulse sensor, a blood oxygen sensor, or one or more electric signal sensors.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims.

### BRIEF DESCRIPTION OF THE DRAWINGS

FIG. 1 is a system diagram for monitoring the health of a person in accordance with some embodiments of the present invention;

FIGS. 2A-2G illustrate a wearable health monitoring device in accordance with some embodiments of the present invention. FIG. 2A: a front perspective view of the wearable health monitoring device clipped onto a wearable article; FIG. 2B: an exploded perspective view of the wearable health monitoring device; FIG. 2C: a front view of the wearable health monitoring device; FIG. 2D: a rear perspective view of the wearable health monitoring device; FIG. 2E: a left perspective view of the wearable health monitoring device;

FIG. 3 illustrates a removable wearable health monitoring device that can be disposed within a swaddle blanket in accordance with some embodiments of the present invention.

FIG. 4 is an exemplary block diagram of a wearable health monitoring device in accordance with some embodiments of the present invention.

FIG. 4A shows exemplary movement sensors suitable for the disclosed wearable health monitoring device.

FIG. 4B shows exemplary bio-vital signal detectors suitable for the disclosed wearable health monitoring device.

FIG. 4C shows exemplary environmental sensors suitable for the disclosed wearable health monitoring device.

FIG. 5A illustrates an exemplified receiving station in accordance with some embodiments of the present invention.

FIG. 5B depicts an exemplified circuit board and display.

FIGS. 6A-6B depict exemplified mobile device displaying interfaces.

FIG. 7 shows a system diagram having various functions including movement log and performance comparison in accordance with some embodiments of the present invention.

FIG. 8 depicts a flowchart in accordance with some embodiments of the present invention; and

FIG. 9A shows exemplary movement patterns that can be recognized in the disclosed system.

FIG. 9B shows exemplary sleeping parameters that can be calculated by the disclosed system.

FIG. 9C shows exemplary environmental parameters that can be calculated by the disclosed system.

FIG. 10 illustrates another system diagram in accordance with some embodiments of the present invention.

### DETAILED DESCRIPTION OF THE INVENTION

The presently disclosed system attempts to address the drawback of insensitive and inadequate monitoring in conventional systems. The disclosed system monitors the health of a person via a wearable device, which monitors baby's bio-vital signals such as respiration rate and body temperature, movement parameters as sleeping position, and ambient conditions. An alert is produced when a criterion for an abnormally is identified. The disclosed system can be used for babies including newborns, infants, and toddlers, as well as people at older ages, such as senior citizens or patients who are prone to respiratory disruptions during sleep.

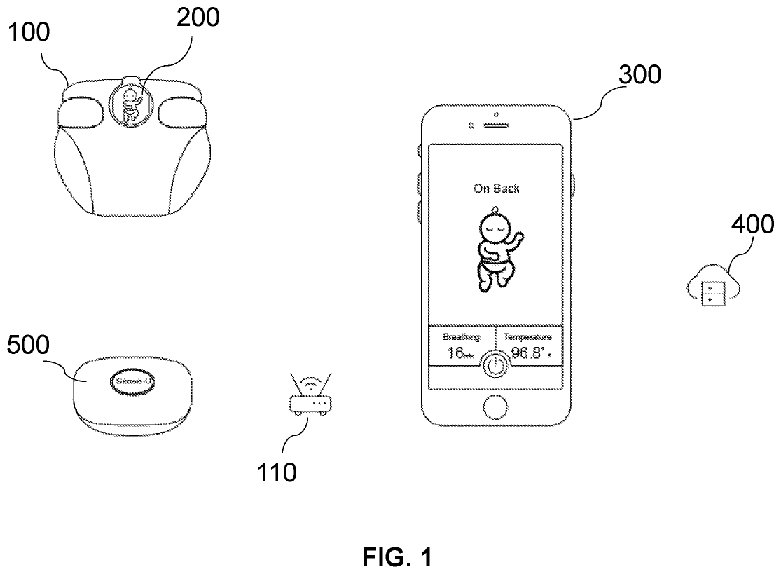

In some embodiments, referring to FIG. 1, a wearable health monitoring device 200 is clipped onto a wearable article 100. The wearable health monitoring device 200 can be in communication with a receiving station 500. The receiving station 500 can be in communication with an internet gateway 110 (e.g., a cable modem, a WiFi router, a DSL modem, etc.). The internet gateway 110 is shown in communication with a computing device such as a mobile device 300. The mobile device 300 can display data that was originally gathered by the wearable health monitoring device 200. Alternatively, the wearable health monitoring device 200 can be in communication with a mobile device 300 directly or via the internet gateway 110 with a receiving station. The internet gateway 110 or the mobile device 300 can be in communication with a cloud server 400. When an abnormal health condition occurs, a parent receives an alert notification, to alert them to immediately check on the status of a baby or infant. The mobile device 300 can display health data collected by the wearable health monitoring device 200.

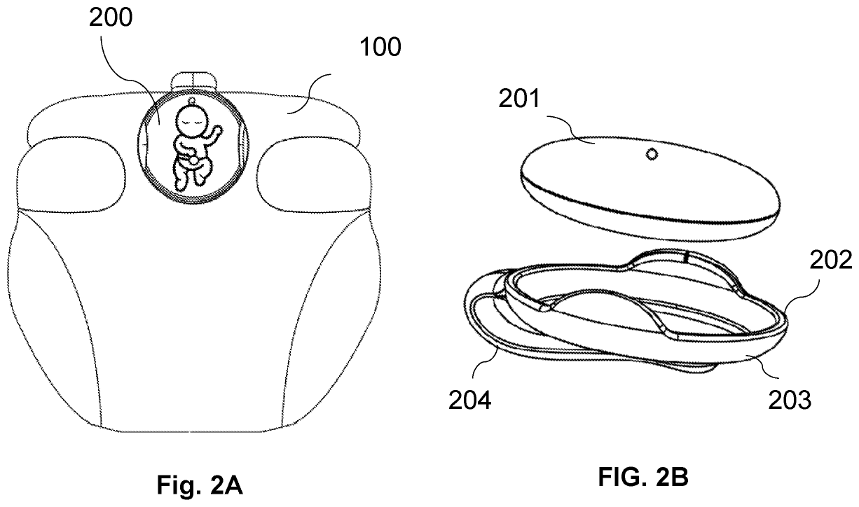

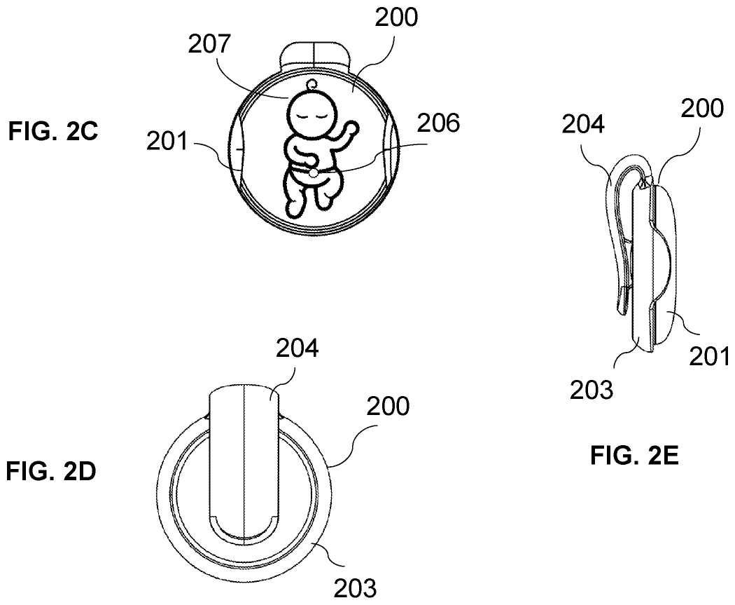

FIG. 2A illustrates an exemplified wearable health monitoring device 200 attached onto or inserted into a wearable article 100 (e.g., a diaper). As shown in FIG. 2B, the exemplified wearable health monitoring device 200 can include a sensor device 201 and a unique securing mechanism 202 including a clip-shaped base 203 and a clip 204. The clip-shaped base 202 is clipped onto the wearable article 100. After pushing the sensor device 201 into the clip-shaped base 202, clipping the wearable health monitoring device 200 onto the wearable article 100, it secures the sensor device 201 in place, creating a tight fastener, which will provide sufficient contact with an infant's abdomen as to detect breathing movement, sleeping position.

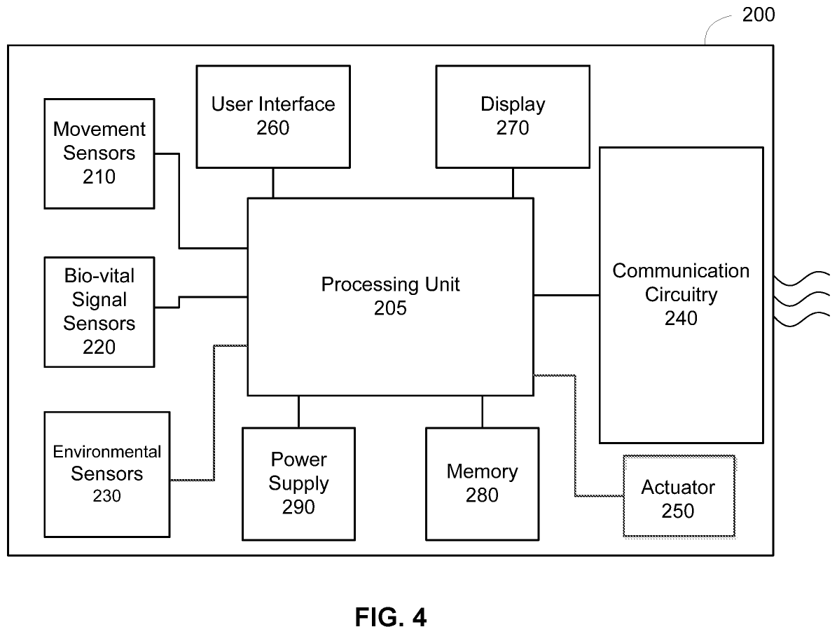

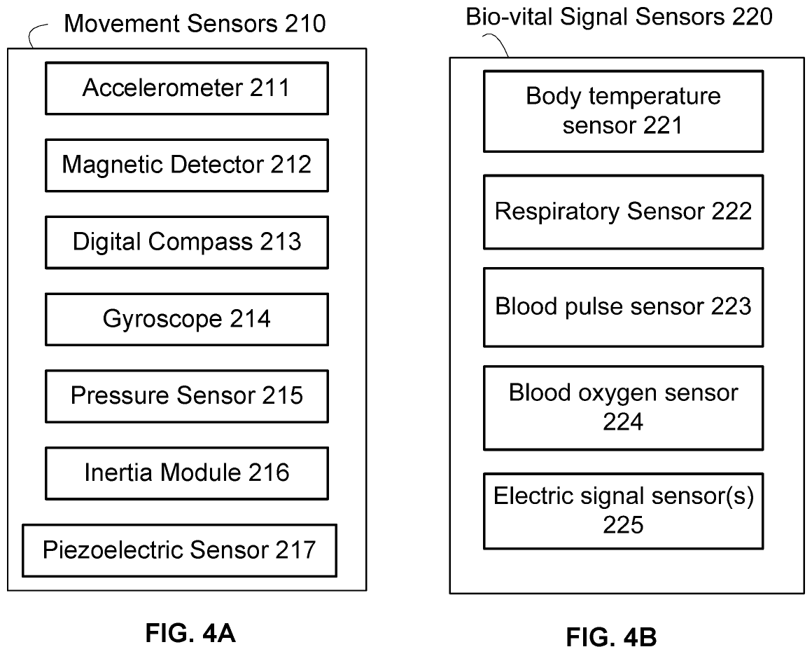

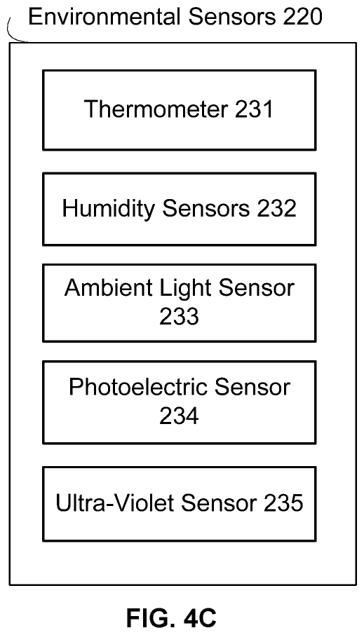

FIGS. 2C, 2D and 2E respectively show the front, the back, and the side views of the wearable health monitoring device 200. The front case of the wearable health monitoring device can have an infant silkscreen 207 illustrating an infant sleeping on his back. It is used to demonstrate the orientation of the wearable health monitoring device together with the recommended sleeping position for an infant. As shown in FIG. 2C, the sensor device 201 can have an infant silkscreen 207 illustrating an infant sleeping on his back, and a temperature contact 206 on one side of the sensor device 201 to measure ambient temperature around the body or directly measure infant's body temperature. To measure body temperature, the device can be worn upside down to touch wearer's skin. As described in detail below, the wearable health monitoring device 200 can include electronics to enable the detection of the movement reading from infant's abdomen. The electrical signals can then be processed by a computer processor (e.g. processing unit 205 in FIG. 4) to generate a respiration rate value and a sleeping position value as well as other related health data.

The sensor device 201 can include on its side a small temperature contact 206. It can be in the form of a small round hole to measure the ambient temperature and humidity level toward the air, and also to use as a user interface with LED lights showing the health information of the infant, or in the form of a metal contact tip to measure body temperature, with the wearable health monitoring device 200 flipped when clipping onto a wearable article. In some embodiments, the sensor device 200 can only measure body temperature while the receiving station 500 measures the ambient temperature.

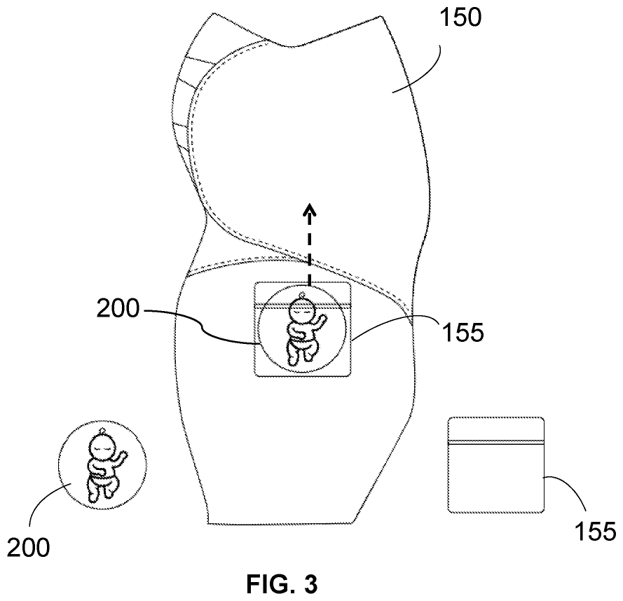

The wearable article 100 can be in the form of a diaper, swaddle blanket, an infant sleeping bag, a wrist band, an elbow or knee pad, or other articles that can be worn by a person. In some embodiments, referring to FIG. 3, a wearable health monitoring device 200 attached onto the wearable article 150 that is in the form of a swaddle blanket that can be wrapped around a person's body. The wearable article 150 can also include a pouch 155 configured to receive the sensor device 201. The pouch 155 can include a zipper that allows the pouch to securely open and close. Additionally, the pouch can include buttons, Velcro, snaps, or other apparatus or useful combination of apparatuses to close the pouch. The sensor device 201, which is receivable into the pouch, can include an outer layer of Velcro or other alignment feature. In particular, the Velcro or alignment feature can be attached to the back of the sensor device 201, which allows the sensor device 201 to be securely fastened to outer strap of the wearable article 150 such that the sensor device 201 is secure and in close contact with the infant's body.

Other details about the above described wearable health monitoring device are disclosed in the commonly assigned U.S. patent application Ser. No. 29/707,085 entitled “Wearable Health Monitor”, filed Sep. 26, 2019, the content of which is incorporated herein by reference.

Referring to FIG. 4, a wearable health monitoring device 200 can be worn on the body of a person such as a baby or an adult for the purpose of health monitoring. The wearable health monitoring device 200 can include movement sensors 210, bio-vital signal sensors 220, and environmental sensors 230, respectively for detecting the activities and the bio-vital signals of the wearing person, and the environment conditions. A processing unit 205 processes the signals detected by movement sensors 210, bio-vital signal sensors 220, and environmental sensors 230 to produce movement data, bio-vital signal data, and environmental data. The wearable health monitoring device 200 can also include one or more actuators 250, a user interface 260, a display 270, a memory 280, and a power supply 290.

FIG. 4A shows examples of the movement sensor 210, which can include one or more of accelerometers 211, magnetic detectors 212, digital compasses 213, gyroscopes 214, pressure sensor 215, inertial module 216, piezoelectric detector 217 and/or other unlisted movement sensors to sense, capture, calculate and record movement data of the object. Different combinations of these movement sensors may be incorporated in the wearable health monitoring devices 200. Even more, all types of detectors, whether listed or unlisted here, that generate data which is representative of the movements of the object, are intended to fall within the scope of the present inventions. The movement sensor 210 can produce signals that represent sleep positions and other movements.

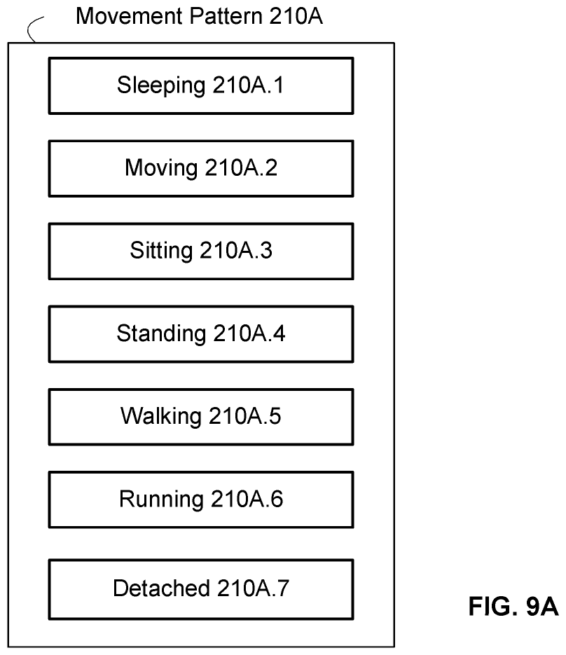

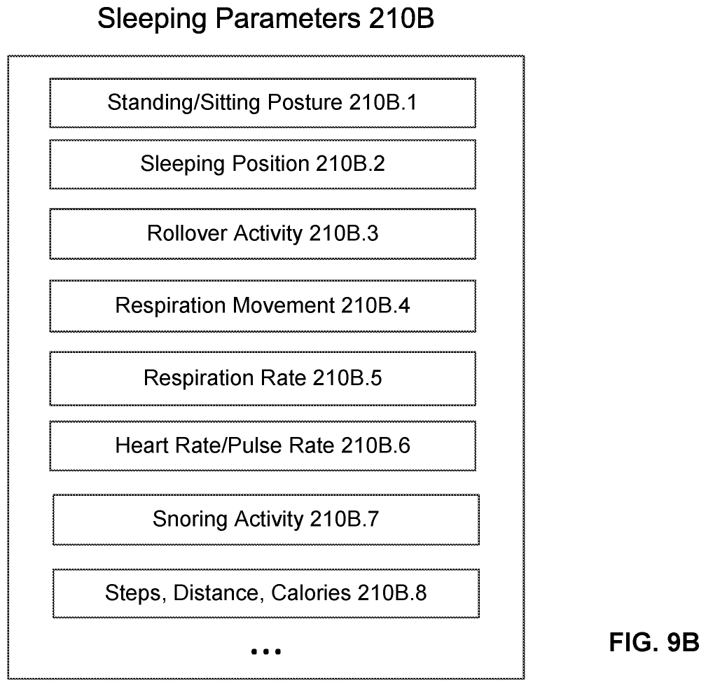

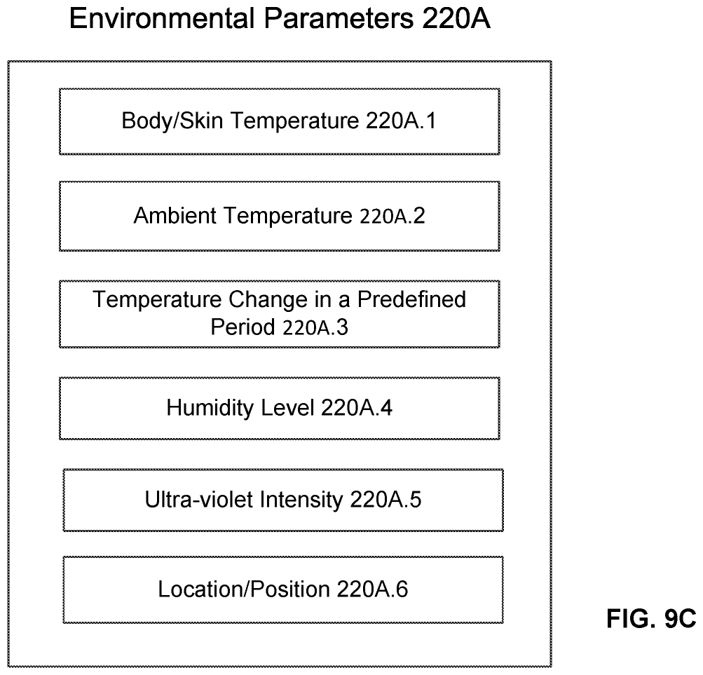

FIG. 4B shows examples of the bio-vital sensors 220, which can include one or more of a body temperature 221, a respiratory sensor 222, a blood pulse sensor 223, a blood oxygen sensor 224, and one or more electric signal sensors 225 (such as ECG or EKG). In some implementations, the respiratory sensor 222 can share a same acceleration sensor as the accelerometers 211. The blood pulse sensor 223 can be implemented as a pressure sensor or sometimes using an acceleration sensor. In the latter implementation, in some implementations, the blood pulse sensor 223 can share a same acceleration sensor as the accelerometers 211. The bio-vital sensors 220 can provide sleep data comprising sleep parameters (210B, FIG. 9B).

FIG. 4C shows examples of the environmental sensor 230, which may include one or more of a thermometer 231 for measuring ambient temperature, one or more humidity sensors 232 that can measure ambient humidity and the humidity in a wearable article such as a diaper, an ambient light sensor 233, a photoelectric sensor 234, and a ultra-violet sensor 235. The environmental sensors may also employ other detectors to provide environmental data which is representative of environmental condition of the wearer. The environmental sensors 230 can provide environmental data comprising environmental parameters (220A, FIG. 9C).

The processing unit 205 processes the captured movement data, the bio-vital signal data, and environmental data. The wearable health monitoring device 200 can also include one or more actuators 250. Exemplified functions of actuators can include emitting a sound or a light, producing vibrations, producing heat, ejecting a fluid (such as chemical or medicine), etc. The actuator 250 can be used to alert a parent when a negative health trend is detected. Also, the actuator 250 can be used to stimulate breathing when a negative trend is detected.

In some embodiments, the wearable health monitoring device 200 can include an accelerometer 211 in the movement sensor 210, a body temperature sensor 221 and a respiratory sensor 222 in the bio-vital sensors 220, a humidity sensor 232 in environmental sensors 230, and a wireless transceiver in a communication circuitry 240. The wearable health monitoring device 200 can be placed on the subject's abdomen to detect respiration rate and sleeping positions. The accelerometer 211 and the respiratory sensor 222 can share a dual acceleration sensor for capture sleeping position as part of the movement data and respiratory signal as part of bio-vital signal data. The body temperature sensor 221 and the humidity sensor 232 provide the temperature reading and humidity level reading respectively. It is believed that babies are most at risk for SIDs when they sleep on their stomach. Accordingly, the accelerometer 211 and the respiratory sensor 222 can produce data that indicate whether the infant is stomach sleeping or sleep on their back as well as the infant's breathing status. In determining the infant's breathing or sleeping position, the wearable health monitoring device 200 can conduct readings for a particular amount of time to avoid false alarms.

The processing unit 205 can include means for processing breathing movement data on-site. Specifically, the processing unit can include all necessary means for processing and filtering the raw movement data and relaying that data to a mobile device 300 or other types of wireless transceivers. For example, the processing unit can convert the raw data into a format that can be broadcast over a particular wireless connection (e.g., Bluetooth, Zigbee, etc.). One will understand, however, that various transmission formats are known in the art and a combination of known transmission formats can be used and remain within the scope of the present invention.

Additionally, the processing unit 205 can receive raw data signal from the movement sensors 210, the bio-vital signal sensors 220, and the environmental sensors 230, further filter out unwanted noise from movement and external sources. As the processing unit processes and filters the received raw data the processing unit can determine at least a respiration rate reading and a sleeping position reading.

In addition to the processing unit 205, the wearable health monitoring device 200 can include a power supply 290, such as a battery, that can power the wearable health monitoring device 200. The battery can be removable or rechargeable. The wearable health monitoring device 200 can also include a visual indicator that indicates when the battery is low on power and need replacing.

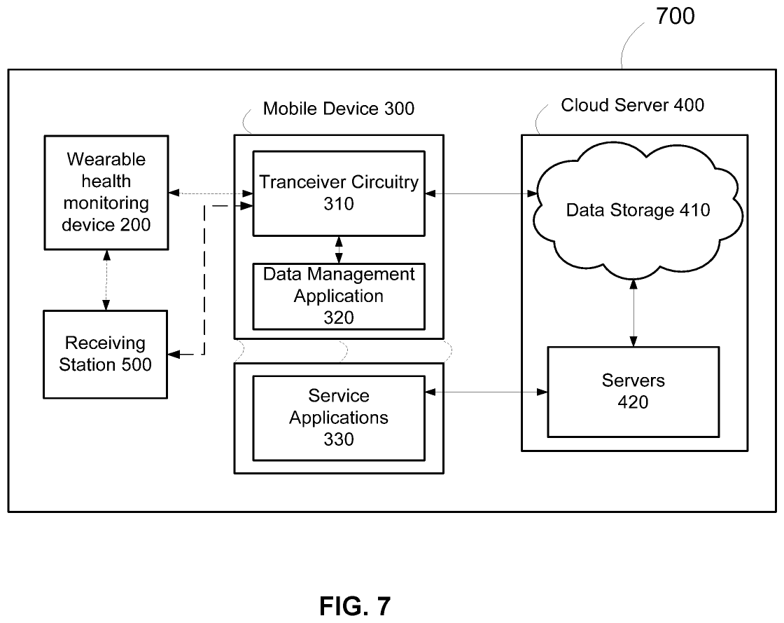

Once the processing unit 205 receives the raw movement data, the processing unit 205 processes the raw data and calculates the respiration movement 210B.4, respiration rate 210B.5 and sleeping position 210B.2 (shown in FIG. 7B), stores in the flash memory 280. The processing unit 205 can then send the processed data to the communication circuitry 240 (shown in FIG. 4) that is also located within the wearable health monitoring device.

The communication circuitry 240 and the processing unit 205 can be located on a print circuit board. In some cases, processing the data at the processing unit 205 before transmitting the data with the communication circuitry 240 can result in significant power savings, as compared to transmitting the raw data. Additionally, processing the data with the processing unit 205 before transmitting the data can improve the data integrity and lower the error rate associated with the data.

The communication circuitry 240 can employ different data transfer methods to build the communication link among the wearable health monitoring device 200, the receiving station 500, and the mobile device 300. Communication links can also include, but not limited to, electronic data link, fire wire, a network cable connection, a serial connection, a parallel connection, USB, or wireless data connection, including but not limited to Bluetooth, Bluetooth Low Energy, WLAN, Zigbee, and proprietary link protocol. Depending upon the implementation, the communication link may employ various communication circuitries 240, operating in one or more modes of transmission and/or receiving. For example, the communication circuitry 240 may include a wireless transceiver, a wireless transmitter, a wired transceiver, and a wired transmitter. The function of the communication link is to transmit and receive data to and from the wearable health monitoring device 200 to a cloud server 400. Depending on the implementation, the communication link may also be coupled to several wearable health monitoring devices to provide a network of sensors all connected to the receiving station 500.

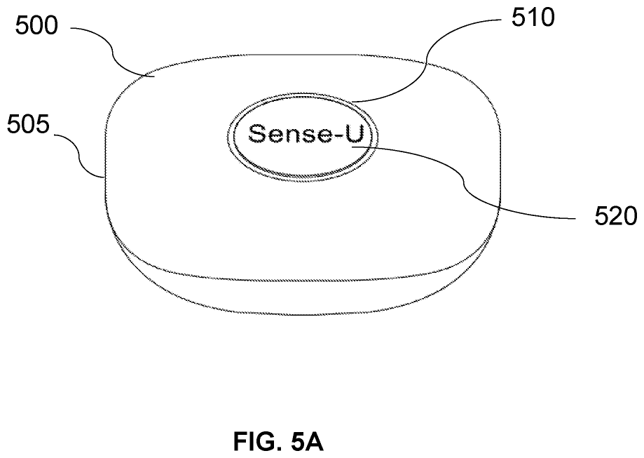

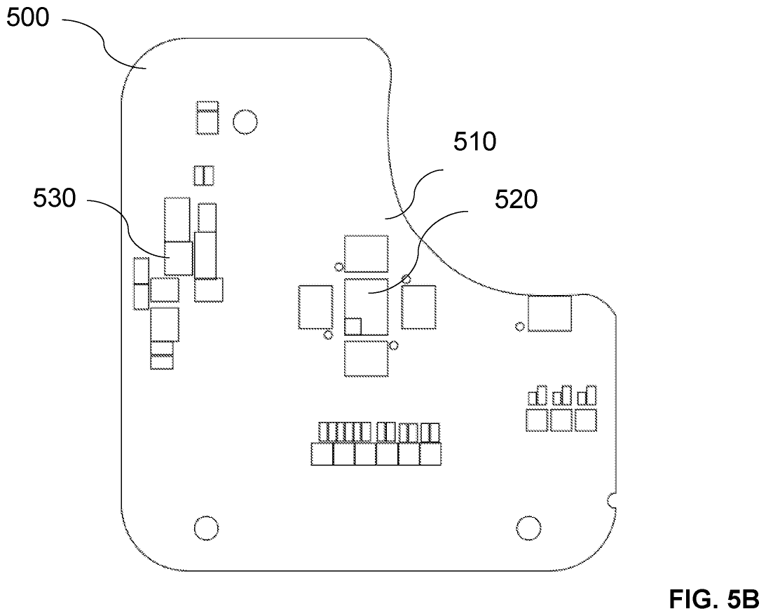

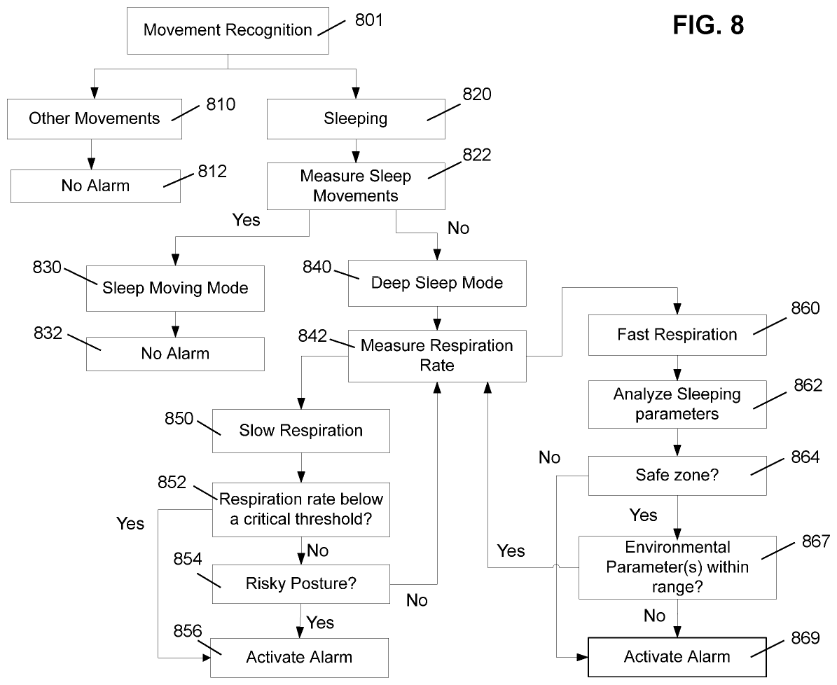

In some embodiments, referring to FIGS. 5A and 5B, a receiving station 500 includes an enclosure 505 with a light-emitting display 510, and a home button 520. The enclosure 505 can be made of a plastic material. The light-emitting display 510 can be implemented by a ring-shaped LED. The light-emitting display 510 allows parents to check the health status of a baby's health easily and receive important health notifications. The home button 520 may make it easy for parents to interrupt a health notification when something happens to their infants. In operation, the receiving station 500 is in communication with the wearable health monitoring device 200, the mobile device 300, and optionally with the cloud server 400. A computer processor 530 can analyze one or more signals or data from the movement sensors 210, the bio-vital signal sensors 220, and the environmental sensors 230 (as described below in relation to FIG. 8).

The receiving station 500 can receive the wireless transmission signal from a wearable health monitoring device 200, or from multiple wearable health monitoring devices 200 attached to different infants. The receiving station 500 can display the health indication data through the light-emitting display 510. The home button 520 on the enclosure of the receiving station may be of the type shown in the drawing and the picture.

Once the data has been processed and transmitted, the receiving station 500 can receive and further process the data. In particular, the receiving station 500 can process the data and detect an abnormal trend in the received movement parameters (shown in FIG. 9B), e.g., respiration data (e.g., slow or fast respiration rate), sleeping position data (e.g., stomach sleeping), or abnormal trend in the received environmental parameters (shown in FIG. 9C), e.g., low temperature, high temperature, temperature variations (temperature drop and temperature increment), high humidity level, or if the receiving station 500 detects a problem within the system (e.g., low battery, poor signal strength, or constant parameters for a certain long time period which indicates the sensor is out of its position, for example, the sensor is fallen off from its originally clamped position, etc.) the receiving station 500 can provide an indication of the problem. For example, the receiving station 500 can sound an alarm, display a notification via the light-emitting display 510 of the receiving station 500, or otherwise send a message.

In some embodiments, referring to FIGS. 1 and 4, after the receiving station 500 has received and further processed the data, the receiving station 500 can transmit the data to an internet gateway 110, such as a WiFi router. Once the data has been received by the internet gateway 110, the data can be transmitted over the internet to a remote computing device 300. The remote computing device 300 can be located within the same local network as the internet gateway 110 such that the data is only transmitted locally and is not transmitted over the internet. Similarly, the wireless transmitter 240 and the processing unit 205 can transmit information directly to the mobile device 300.

In some embodiments, the data can be transmitted to a mobile device 300 directly. In the case if the mobile device 300 detects an abnormal trend, the mobile device 300 can provide an indication of the problem. For example, the mobile device 300 can sound an alarm, display a notification on the screen of the mobile device 300, or otherwise send a message.

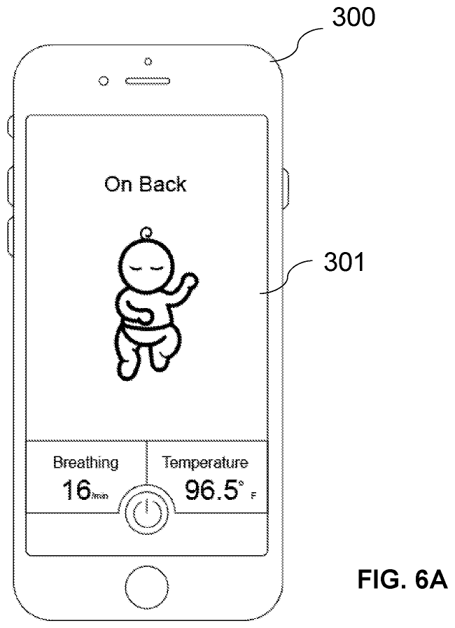

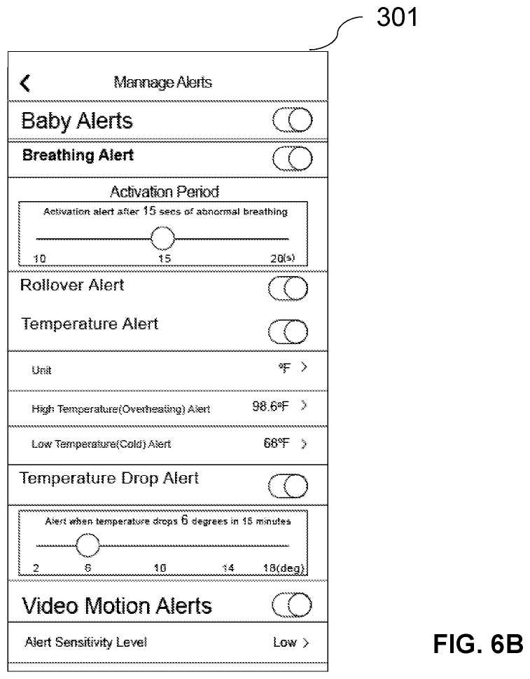

In some embodiments, the mobile device 300 can display analyses of the received movement data, bio-vital signal data, and the environmental data. For example, the mobile device 300 can include a user interface 301 that displays real-time respiration rate, sleeping position and temperature of the infant (shown in FIG. 6A). Similarly, the mobile device 300 can show a graph tracking the respiration rate, or temperature of an infant over time. Additionally, the user interface 301 can customize the settings of wearable health monitoring device and the alert threshold (shown in FIG. 6B). In general, the mobile device 300 can utilize the received information to display a variety of health data. In this way, when an abnormal health reading happens, a parent can receive an alert notification and further determine the urgency of the notification. The mobile device 300 can be in the form of a smart phone, a tablet computer, a personal computer, a laptop, or a customized computing device.

In some embodiments, a user can configure the receiving station 500's response to a particular alarm or to alarms in general. For example, a user can silence all alerts by tapping the ON/OFF button on the home screen of the application (FIG. 6A). Similarly, a user can also configure the receiving station 500 to only indicate an alarm if the alarm is enabled (FIG. 6B). Further, a user can configure the receiving station 500 to only indicate an alarm if certain conditions are met, e.g., temperature falling out of the range (FIG. 6B). Similarly, upon receiving an alert generated by the wearable health monitoring device 200, or upon receiving health data that demonstrates a negative trend, the mobile device 300 can also be configured to indicate an alert. For example, the mobile device 300 can sound an audible alarm, vibrate, or generate a visual alert. It should be understood that the presently disclosed systems and methods are compatible with a multitude of methods for receiving station 500 and the mobile device 300 to alert a user.

In addition, false alarms cause anxiety and unnecessary fear but many babies naturally hold their breath for short periods of time, causing slow and fast respiration rates. Since this can be a normal occurrence, the base station 500 can include a delayed alarm mechanism. The mobile device 300 can adjust the activation period (as shown in FIG. 6B) to reduce or even avoid these false alarms.

In some embodiments, the high temperature threshold and the temperature increment in a predefined period can be used to monitor the sign of baby overheating and the low temperature threshold and the temperature drop parameters can be used to monitor the sign of getting cold.

The wearable health monitoring device 200 can integrate with a combination of movement sensors 210 as shown in FIG. 4A, and a combination of the environmental sensors 230 as shown in FIG. 4B. In this implementation, the mobile device 300 can alert a user to information of interest, including abnormal health trends.

In some embodiments, referring to FIG. 7, a health monitoring system 700 includes a wearable health monitoring device 200, a mobile device 300, and a cloud server 400 comprising a data storage 410 and servers 420. The mobile device 300 can include a transceiver circuit 310, data management applications 320, and service applications 330. The cloud server 400 can access a historical record of health recordings, and video clips. For example, a parent of an infant can access a historical record of the infant's breathing and related health data and provide the record to the infant's doctor. The remote cloud server 400 can access a historical record of health recordings. For example, a parent of an infant can access a historical record of the infant's breathing and related health data and provide the record to the infant's doctor. The accessed historical record can be stored by the cloud server 400, the receiving station 500, the mobile device 300, or some other web-based storage cache. Optionally, the health monitoring system 700 can include a receiving station 500. As disclosed above in FIGS. 5A and 5B, the wearable health monitoring device 200 can communicate with the receiving station 500, which can in turn communicate with the mobile device 300 and/or the cloud server 400.

The mobile device 300 can receive the wireless signal from the wearable health monitoring device 200 directly, or from the server 400. The mobile device 300, as shown in FIGS. 6A and 6B above, can then display the data on its screen, including real-time breathing data, visualized sleeping positions, temperature, etc. It can also distinguish between the potentially multiple wearable health monitoring devices 200.

In some embodiments, a health monitoring system can include the wearable health monitoring device 200, the receiving station 500, the mobile device 300, and the cloud server 400. In this implementation, the wearable health monitoring device 200 can communicate with the receiving station 500, which can in turn communicate with the cloud server 400, and/or the mobile device 300. Both the receiving station 500 and the mobile device 300 can alert a user to information of interest, including abnormal health trends.

Furthermore, in some embodiments, a health monitoring system can include the wearable article 100, the wearable health monitoring device 200, the receiving station 500, the mobile device 300, and the cloud server 400. In this implementation, the wearable article 100 integrated with the wearable health monitoring device 200, forms a smart diaper and can communicate to the receiving station 500 through a wireless protocol. The receiving station 500 can then transmit information to a remote cloud server 400 of interest.

An exemplified process associated with the presently disclosed health monitoring system is now described. First, the disclosed health monitoring system monitors the proper attachment of the wearable health monitoring device to the wearing person, such as an infant. If it is found that the wearable health monitoring device is not properly attached or disposed in a wearable article (such as a diaper or a swaddle blanket), the health monitoring system emits an warning to allow a parent, a care taker, or the wearing person to fix the attachment of the wearable health monitoring device.

Referring to FIGS. 8 and 9A, using the monitoring the health of an infant as an example, the movement pattern of an infant wearing the wearable health monitor device is recognized (step 801) based on the movement data detected by one or more movement sensors in the wearable health monitoring device 200. Examples of movement patterns 210A (FIG. 9A) that can be recognized by the disclosed health monitoring system can include sleeping 210A.1, moving 210A.2, sitting 210A.3, standing 210A.4, walking 210A.5, running 210A.6, and the wearable health monitoring device detached 210A.7.

If it is determined that the infant is not sleeping 210A.1 but in one of other movement patterns such as moving 210A.2, sitting 210A.3, standing 210A.4, walking 210A.5, running 210A.6, or detached modes 210A.7 (step 810), no alarm will be activated (step 812) related to the prevention of Sudden Infant Death Syndrome. It should also be noted that movement data can be used to analyze and produce alarms for other movement behaviors. For example, when the movement data show that a baby is climbing the guardrail of a babe bed, an alarm signal will be delivered to the babe's guardian.

If it is determined that the infant is sleeping 210A.1 (step 820), sleep movements are measured (step 822). If the amount of movements during sleep is over a threshold, the infant will be considered to be in a sleep moving mode (which can be considered as an instance of the moving mode 210A.2) (step 830). The infant is determined to be in a safe state and no alarm will be produced (step 832).

If the amount of movements is below a threshold, the infant will be considered to be in a deep sleep mode (step 840), the disclosed health monitoring system conducts a measurement of one or more bio-vital signals such as the respiratory rate of the infant (step 842) using one or more of the bio-vital signal sensor (220 in FIG. 4B).

If the measured respiration rate is below a respiration threshold, the infant is determined to be in a slow respiration mode (step 850). The disclosed health monitoring system checks if the respiration rate is below a critical threshold (step 852). If it is, which includes the situation of a stop of breathing, an alarm is produced (step 856). Moreover, a threshold amount of time can be allowed for the health readings to return to a normal level. If the respiration rate is above a critical threshold, the disclosed health monitoring system further determines the sleeping posture (210B.2 in FIG. 9B) of the infant (step 854) using the movement data from one or the movement sensors such as an accelerometer (211, FIG. 4A) disposed next to the stomach of the infant. If the detected infant posture is sleeping on stomach, then the disclosed health monitoring system can conclude that the abnormal respiration rate may be related to the stomach sleeping. Therefore, the disclosed health monitoring system produces an alarm signal indicating that the slow respiration rate alarm which might be related to the stomach sleeping (step 856). The alarm signal can be in one or a combination of forms such as audible, visual, vibration, or an electronic text, etc. If it is detected that the infant is sleeping in a safe posture (on the back or a side), the health monitoring system returns to measuring and continuing to monitor respiratory rate (842).

Exemplified sleeping parameters are shown in FIG. 9B. The calculated movement parameters 210B include, but are not limited to standing/sitting posture 210B.1, sleeping position 210B.2, rollover 210B.3, breathing movement 210B.4, breathing/respiratory rate 210B.5, heart rate/pulse rate 210B.6, snoring 210B.7, steps/di stance/calories 210B.8, and sleep quality 210B.9.

If the measured respiration rate (in step 842) is above a respiration threshold, the infant will be considered to be in a fast respiration mode (step 860). The health monitoring system analyzes the corresponding sleeping parameters (FIG. 9B) (step 862), which can include the absolute value of the fast respiration (over another safe respiration threshold) and the period of time that the infant stays at such fast respiration (for an extended period long than a threshold period). It is then determined whether the respiration behaviors are within a safe zone (step 864). If the respiration behaviors do not meet criteria for a safe zone (e.g. respiration is overly fast or for a longer enough period of time) (step 864), the health monitoring system activates alarm (step 869).

Next, if the respiration behaviors meet criteria for a safe zone, the health monitoring system can further measure one or more environmental parameter and determines whether an environmental parameter measured is out of range (step 867). Examples of environmental parameters 220A (FIG. 9C) can include, but are not limited to body/skin temperature 220A.1, ambient temperature 220A.2, temperature change in a predefined period 220A.3, humidity level 220A.4 (that can include measurement data on ambient humidity and the humidity in a wearable article such as a diaper), ultra-violet intensity level 220A.5, and location/position 220A.6. If one or more environmental parameters are out of safe range (e.g. the ambient temperature or the ambient humidity over respective desirable ranges), the health monitoring system produces an alarm (step 869) to alert the parents to check on the fast breathing status. If one or more environmental parameters are within safe range (e.g. the ambient temperature or the ambient humidity within respective desirable ranges), the health monitoring system returns to measuring and monitoring respiration rate (step 842).

Throughout the process, the health monitoring system continues monitor movement patterns (step 801). If the infant wakes up and exhibit movement behaviors other than sleep (step 810), the health monitoring system considers the infant to be in a safe state (step 812).

It should be noted that although part of the above process is described using an infant as an example, the disclosed process and system are applicable to persons of other ages. The described operation steps are consistent with persons of older age wearing the disclosed wearable health monitor device. An example of an elderly person that may require health monitoring is someone who has Alzheimer's disease. The alarm signals can be sent to his or her guardian or caretaker. The disclosed health care system can be used to detect early symptoms of Alzheimer's disease and Parkinson's disease in a person wearing the disclosed wearable heath monitoring device. The disclosed health care system can also be used to help patients to delay, slow down, or prevent the development of Alzheimer's disease and Parkinson's disease. The disclosed health care system can also be used to monitor, and/or prevent, and alert respiration issues of elderly persons. Another example for a need for the disclosed health monitoring system is someone having asthma, wherein the monitoring of respiration rate and blood pulse can be valuable for early intervention. The third example for a need for the disclosed health monitoring system is someone having Sleep-disordered breathing (SDB), SDB can range from frequent loud snoring to Obstructive Sleep Apnea (OSA) a condition involving repeated episodes of partial or complete blockage of the airway during sleep, wherein the monitoring of respiration rate and notification of slow respiration rate can be valuable for early intervention. One more example for a need for such system is someone has a pneumonia or a fever, wherein the monitoring of respiration rate and body temperature and notifications of abnormal respiration rates and abnormal temperatures can be valuable for early intervention.

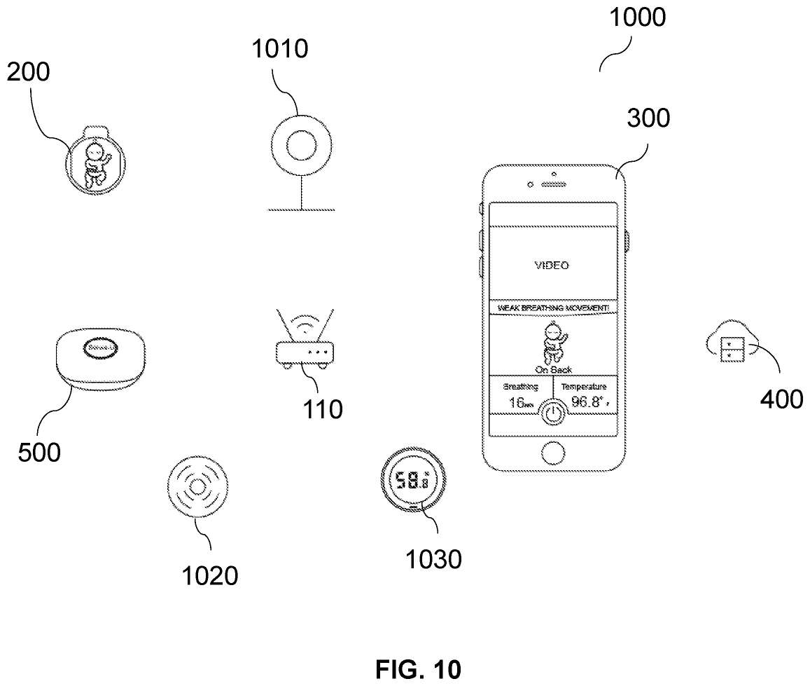

The above described operation steps can be implemented by one or multiple devices including the wearable health monitoring device 200 (FIGS. 1, 4, 7, and 10), the mobile device 300 (FIGS. 1, 6A, 6B, 7, 10), the receiving station 500 (FIGS. 5A, 5B, 10), the remote cloud server 400 (FIGS. 1, 7, and 10), or other devices compatible with the presently disclosed system. For example, the analyses and the recognition of the wearing person's movement can be conducted on the wearable health monitoring device 200 (FIGS. 1, 4, 7, and 10), while further analyses can be conducted on the mobile device 300 (FIGS. 1, 6A, 6B, 7, 10), the receiving station 500 (FIGS. 5A, 5B, 10), or the remote cloud server 400 (FIGS. 1, 7, and 10). In some embodiments, all the analysis steps in FIG. 8 can be conducted on the mobile device 300 (FIGS. 1, 6A, 6B, 7, 10), or the receiving station 500 (FIGS. 5A, 5B, 10), or the remote cloud server 400 (FIGS. 1, 7, and 10).

In general, an abnormal reading can consist of abnormal respiration rate reading that falls out of a predefined range. Additionally, abnormal readings can also represent a temperature reading or temperature variation that fall out of a predefined range. Further, abnormal readings can also consist of a stomach sleeping position or a bad breathing movement waveform. It should be understood that the described abnormal readings are not meant as an exhaustive list of the abnormalities that the presently disclosed systems and methods can identify and compensate for.

It should be noted that the above disclosed operation steps can be implemented using machine learning. A deep learning model can be trained by movement data, bio-vital signal data, and environmental parameters data and known conditions of the wearing person. The trained model can be used separately or in combination with the flowchart disclosed above to automatically determine the state of the wearing person and the need for generating an alarm.

FIG. 10 illustrates an infant monitoring system 1000 for monitoring the health of an infant. In particular, a mobile device 300 is in communication with a variety of infant monitoring devices, for example, a wearable health monitoring device 200, a video monitor 1010, a smoke and carbon monoxide detector 1020, and a room thermometer 1030. In some embodiments, an infant monitoring system can include one or more of these devices in combination. For example, the wearable health monitoring device 200 may detect an abnormal health reading. In response to the reading, the mobile device 300 can request a video clip transmitted from a video monitor 700, smoke and carbon monoxide readings from a smoke and carbon monoxide detector 1020, or temperature/humidity readings from a room thermometer 1030. The video monitor 1010 can generate detect movements of the wearing person and its output signals can be sued to generate video motion alerts (shown in FIG. 6B). In this way, a parent can receive a notification that an abnormal health reading has occurred, while at the same time receiving more data of the infant to determine whether the notification is an emergency.

In some embodiments, a video monitor 1010 can capture videos of the infant and streams them to the smart device 300 via a router device 110. When an abnormal health reading happens, a parent can receive an alert notification, together with a corresponding video clip, to further determine the urgency of the notification.

Accordingly, FIGS. 1-10 provide a number of components, schematics, and mechanisms for wirelessly monitoring the health of an infant. In particular, a wireless wearable health monitoring device 200 communicates health data from an infant to a receiving station 500. The receiving station 500 can then be used to monitor the infant's health or to alert an individual to a negative trend in the infant's health. Additionally, false alarms can be detected, and in some cases prevented, by analyzing trends in the received health data.

The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for monitoring health of a person by a health monitoring system, comprising:
determining, based on movement data, if a person wearing a wearable health monitoring device is sleeping or not, wherein the movement data is produced by one or more movement sensors in the wearable health monitoring device;
when the person is determined to be sleeping, measuring sleep movements of the person to determine whether the person is in a deep sleep mode;
measuring one or more bio-vital signals of the person and at least one of a posture of the person or one or more environmental parameters when the person is determined to be in the deep sleep mode, wherein the one or more bio-vital signals are produced by one or more bio-vital signal detectors in the wearable health monitoring device; and
automatically producing an alarm signal if a predetermined criterion is met based on the one or more bio-vital signals and at least one of a posture of the person, or the one or more environmental parameters.

2. The method of claim 1, wherein the one or more bio-vital signals include a respiration rate, the method further comprising:
automatically determining by a health monitoring system whether the person has a slow respiration or a fast respiration by comparing the respiration rate to a predetermined respiration threshold,
wherein the health monitoring system includes the wearable health monitoring device.

3. The method of claim 2, further comprising:
when the person is determined to have a slow respiration, automatically producing the alarm signal if the respiration rate is below a critical respiration threshold.

4. The method of claim 2, further comprising:
when the person is determined to have a slow respiration, automatically determining a posture of the person based on measurement by an accelerometer sensor in the wearable health monitoring device,
wherein the alarm signal is produced if the person is determined to be sleeping on stomach based on the posture of the person.

5. The method of claim 2, further comprising:
when the person is determined to have a fast respiration, automatically determining whether respiration behaviors are within a safe zone based on an absolute value of the respiration rate and a period of time within which the respiration rate is above the predetermined respiration threshold; and
automatically producing an alarm signal if the respiration behaviors are outside of the safe zone.

6. The method of claim 5, wherein the respiration behaviors are outside of the safe zone when the respiration behaviors are above a safe respiration threshold, or the person is determined to have a fast respiration for an extended period long than a threshold period, or a combination thereof.

7. The method of claim 2, further comprising:
when the person is determined to have a fast respiration, measuring one or more environmental parameters;
automatically determining by a health monitoring system whether the one or more environmental parameters are within respective desirable ranges, wherein the health monitoring system includes the wearable health monitoring device,
wherein the alarm signal is produced if the one or more environmental parameters are determined to be outside of respective desirable ranges.

8. The method of claim 76, wherein the one or more environmental parameters include ambient temperature or humidity.

9. The method of claim 1, wherein the person is an infant, baby, or toddler.

10. The method of claim 9, wherein the wearable health monitoring device is attached to or removably disposed in a wearable article worn adjacent to the baby's abdomen.

11. The method of claim 1, wherein the one or more movement sensors in the wearable health monitoring device includes one or more of accelerators, a magnetic detector, a digital compass, a gyroscope, a pressure sensor, an inertia module, or a piezoelectric sensor.

12. The method of claim 1, wherein the one or more bio-vital signal detectors in the wearable health monitoring device include one or more of a body temperature sensor, a respiratory sensor, a blood pulse sensor, a blood oxygen sensor, or one or more electric signal sensors.

13. A health monitoring system, comprising:
a wearable health monitoring device comprising one or more movement sensors configured to produce movement data of a person that wears the wearable health monitoring device, wherein the wearable health monitoring device comprises one or more bio-vital signal detectors configured to produce one or more bio-vital signals; and
one or more computer processors configured to determine, if the person is sleeping or not based on movement data, wherein the one or more movement sensors are configured to measure sleep movements of the person when the person is determined to be sleeping, wherein the one or more computer processors are configured to determine whether the person is in a deep sleep mode,
wherein when the person is determined to be in the deep sleep mode, the one or more computer processors are configured to produce an alarm signal if a predetermined criterion is met based on the one or more bio-vital signals and at least one of a posture of the person.

14. The health monitoring system of claim 13, wherein the one or more bio-vital signals include a respiration rate,
wherein the one or more computer processors are configured to automatically determine whether the person has a slow respiration or a fast respiration by comparing the respiration rate to a predetermined respiration threshold.

15. The health monitoring system of claim 14, wherein when the person is determined to have a slow respiration, the one or more computer processors are configured to produce the alarm signal if the respiration rate is below a critical respiration threshold.

16. The health monitoring system of claim 14, wherein the wearable health monitoring device includes an accelerometer,
wherein when the person is determined to have a slow respiration, the one or more computer processors are configured to automatically determine a posture of the person based on measurement by the accelerometer,
wherein the alarm signal is produced if the person is determined to be sleeping on stomach based on the posture of the person.

17. The health monitoring system of claim 14, wherein when the person is determined to have a fast respiration, the one or more computer processors are configured to automatically determine whether respiration behaviors are within a safe zone based on an absolute value of the respiration rate and a period of time within which the respiration rate is above the predetermined respiration threshold,
wherein the one or more computer processors are configured to automatically produce an alarm signal if the respiration behaviors are outside of the safe zone.

18. The health monitoring system of claim 17, wherein the respiration behaviors are outside of the safe zone when the respiration behaviors are above a safe respiration threshold, or the person is determined to have a fast respiration for an extended period long than a threshold period, or a combination thereof.

19. The health monitoring system of claim 14, further comprising:
one or more environmental sensors configured to produce one or more environmental parameters,
when the person is determined to be in the deep sleep mode, the one or more computer processors are further configured to produce an alarm signal if a predetermined criterion is met based on the one or more bio-vital signals and at least one of a posture of the person or one or more environmental parameters.

20. The health monitoring system of claim 19, wherein when the person is determined to have a fast respiration, one or more environmental sensors are configured to measure one or more environmental parameters,
wherein the one or more computer processors are configured to automatically determine whether the one or more environmental parameters are within respective desirable ranges,
wherein the one or more computer processors are configured to automatically produce the alarm signal if the one or more environmental parameters are determined to be outside of respective desirable ranges,
wherein the one or more environmental parameters include ambient temperature or humidity.

21. The health monitoring system of claim 13, wherein the person is an infant, baby, or toddler, wherein the wearable health monitoring device is attached to or removably disposed in a wearable article worn by and in contact with the baby's abdomen.

22. The health monitoring system of claim 13, wherein the one or more movement sensors include one or more of an accelerator, a magnetic detector, a digital compass, a gyroscope, a pressure sensor, an inertia module, or a piezoelectric sensor.

23. The health monitoring system of claim 13, wherein the one or more bio-vital signal detectors include one or more of a body temperature sensor, a respiratory sensor, a blood pulse sensor, a blood oxygen sensor, or one or more electric signal sensors.
